(19) United States
(12) Patent Application Publication
(10) Pub. No.: US 2020/0214621 A1
(43) Pub. Date: Jul. 9, 2020

(54) PRESSURE ULCER PREVENTION TOOL

(71) Applicant: Thomas Jefferson University, Philadelphia, PA (US)

(72) Inventors: Mark Keroles, Rancho Palos Verdes, CA (US); Daniel S. Choi, Philadelphia, PA (US); Vinayak Rajandran, Hockessin, DE (US); Adam Michael Hecht, Ridgewood, NJ (US)

(73) Assignee: Thomas Jefferson University, Philadelphia, PA (US)

(21) Appl. No.: 16/648,641

(22) PCT Filed: Sep. 7, 2018
(86) PCT No.: PCT/US2018/051359

§ 371 Date: Mar. 18, 2020

Related U.S. Application Data
(60) Provisional application No. 62/560,420, filed on Sep. 19, 2017.

(51) Int. Cl. A61B 5/00; G08B 21/24; A61G 7/057; G09G 5/22; G16H 40/67

(52) U.S. Cl. CPC A61B 5/447; G08B 21/24; A61B 5/6831; A61B 5/6892; A61B 5/746; G09G 2354/00; A61G 7/057; G09G 5/22; G16H 40/67; A61B 2562/0219; A61B 5/7445

(57) ABSTRACT

Systems and methods for detecting patient movement and ensuring compliance with patient turning, comprising strap having at least a horizontal and one or more sensors positioned on the horizontal component; a display comprising a processor and a database, configured to receive information from the one or more sensors and to generate a timer to ensure patient turning at a predetermined time.

### FIELD OF THE INVENTION

This application is generally related to devices and systems for monitoring patients and preventing formation of pressure ulcers.

### BACKGROUND OF INVENTION

Pressure sores are areas of damaged skin caused by staying in one position for too long. They commonly form where bones are close to the skin, such as the ankles, back, elbows, heels and hips. A patient is at risk if they are bedridden, use a wheelchair, or are unable to change their position. Pressure sores can cause serious infections, some of which are life-threatening. For patients who have limited mobility, including those in hospital settings, emergency room, home care settings, nursing homes, and the like, such sores are highly problematic.

Sores can be prevented through a time intensive protocol, including keeping skin clean and dry, changing position every two hours, using pillows and products that relieve pressure, and movement of the body, including removal of pressure, such as by standing and walking, to increase blood flow. If sores occur, a variety of treatments are available, though prevention is best. If sores do occur, advanced sores are slow to heal, so early treatment is best.

Particular problems in both long term care and in emergency care is that one of the most beneficial steps to prevent pressure ulcers is by turning the patient. This includes physically and, often, manually moving the patient from one position to another. Frequently, the patient is turned from a “left side” to a “right side” to ensure that pressure is on only one side at a time. However, nurses do not log patient turns immediately after they are done and this can lead to confusion about how to turn a patient and when to do so. Nurses may have to rely on their memory for when a patient needs to be turned when logging is not accurately performed. Nurses do not know about a patient's position and movements in bed that effect pressure ulcer risk. If a patient has prematurely aborted a turn, the nurse does not know when that occurred. Also, there is no central place to look to see the turn status of every patient. Family members do not have concrete evidence about their patient's turns. And family members who are turning their patient do not have feedback if the turn was done correctly.

These problems can be somewhat alleviated by some existing beds, that inflate and deflate to change the pressure positions on a patient. However, these are not suitable for all patients, nor are they easily provided in all health-care settings due to cost, size, or electrical connection, as a few examples of their limitations.

Therefore, new devices are necessary to alleviate these issues and provide for tools to assist care givers in preventing pressure ulcers.

### SUMMARY OF INVENTION

A pressure ulcer prevention tool (the device) in accordance with an aspect of the invention may comprise four components: (1) one or more sensors, for example two sensors (which can, for example, comprise Inertial Measurement Units (IMUs)) which may each include one or more of accelerometers, gyroscopes, and magnetometers) mounted to the head of the bed using a specialized strap that helps transfer motion from the patient to the sensors (although the invention is not limited to the use of such a specialized strap), (2) a specialized algorithm running on a processor of a controller of the device that takes the data collected by the IMUs, stores it in a memor of the controller and produces actionable information for the prevention of pressure ulcers, (3) an intuitive bed-mounted display that delivers information to the healthcare provider in multiple forms, for example three forms (to be discussed further below), each tailored to deliver information at varying distances and to reinforce the information delivered by the other forms, and (4) a central repository of information, referred to below as a “dashboard”, that allows a health care professional to view the pressure ulcer related patient information from a remote location. The memory also stores a program or other logic that effects the algorithm by running the program on the processor. Each device also preferably includes a transceiver to communicate, e.g., wirelessly, with the dashboard, which itself has a transceiver for receiving data wirelessly. Alternatively, the device(s) can be configured to connect to the dashboard in a wired manner, e.g., over the hospital's wired Internet connection.

The sensors and strap used in the pressure ulcer prevention tool in accordance with an aspect of the invention are preferably durable and easily cleaned. The strap is preferably made up of one horizontal strip of material and two vertical strips of material, which vertical strips are attached to the horizontal strip, although the vertical strips are not necessary, for example if the horizontal strap is affixed to the bed by an adhesive. The horizontal strip of the strap is adjustable so that the strap can accommodate different sized mattresses. It is secured in the horizontal position using a “buckle with slider bar”. Alternatively the horizontal strip of fabric can contain elastic material on the underside that allows the strap to be stretched in the horizontal axis and will squeeze the bed once positioned. This design resembles the article of clothing called overalls. We will refer to this design as the “overalls” design from here on. The overalls design helps the healthcare professional position the strap and sensors in the correct location on the bed by limiting the vertical position that the strap can be positioned in. This overalls design is also key to the strap being easy to clean. Because it is made of strips of durable material (for example, nylon sheets) instead of being a solid cover that goes over the head of the bed, a nurse or member of the sanitation crew will be able to easily clean the top and underside of the strap.

The algorithm that processes gyroscope and accelerometer information collected by the sensors performs a number of tasks to help a health care professional prevent pressure ulcers.

In a preferred embodiment, the algorithm processes information from the Z-Axis and Y-Axis of the gyroscopes of each sensor that utilizes a gyroscope. Using the information from the gyroscope Z-Axis the patient's horizontal position in the bed is determined. Using the gyroscope Y-Axis the inclination angle of the patient/bed is determined. Using the X-Axis, Y-Axis, and Z-Axis of the accelerometer the patient's instantaneous motion in the bed is determined. A reason for using the gyroscope to determine position is because the gyroscope is able to detect when a patient changes position and how long the patient is in that new position. In contrast, the accelerometer can only sense instantaneous changes in position and cannot sense that a patient is sustaining a changed position. It could be assumed that the patient is sustaining a position using the accelerometer, but applicants have found that using the concrete information from the gyroscope is more accurate than using the accelerometer and making an assumption. The reason the accelerometer is used to detect patient motion instead of the gyroscope is because the accelerometer can better detect “micro motion.”

In its simplest form, the algorithm takes the sensor data and derives patient position and motion information from it. The healthcare provider can then make decisions on how to alter patient treatment. In a more advanced form, the algorithm can synthesize information from all of the sensors into treatment recommendations for the health care provider.

Information collected by the sensors is presented on both the bed-mounted display and a “Dashboard,” as described below. In a preferred embodiment, the bed-mounted display presents only directly actionable information for pressure ulcer prevention, while the Dashboard, in addition to the display information, may also present historical patient information and statistics about the patient's pressure ulcer prevention.

According to an aspect of the invention, the user interface of the bed-mounted display is organized into the “main screen”, multiple “secondary screens” and an “edit menu”. If the patient turn is not overdue then the main screen will display a timer that counts down (indicating how long until the next required movement of the patient) and a circular graph that depletes and changes color as the timer counts down. The color graph will, for example, progress from green to orange to red as the graph depletes. If the patient turn is overdue the main screen will change to, for example, have a solid red circular graph that slowly pulsates. It will also display text that alerts the health care provider that the patient turn is overdue and will have a timer that counts up to indicate by how long the turn is overdue. The secondary screens can be accessed by rotating a bezel of the bed mounted display. In a presently preferred embodiment, there are 3 secondary displays that show, respectively, average time between patient turns, number of hours the patient has been inclined in the past 24 hours, and percentage of time patient has spent in the center, left turn, and right turn positions.

The Dashboard can be accessed from desktop computers as well as mobile devices. For example, the user interface of the Dashboard can be configured to allow the health care provider to display information from all occupied beds in the hospital, a specific hospital floor, or an individual patient. According to one aspect of the invention, the Dashboard is configured to display all of the information that the bed-mounted display shows, along with other historical and statistical data based on the needs of the user. In order to identify the bed from which the information is derived, each bed has an electronic identification (ID), allowing the desktop computer, or mobile device, to distinguish information for each bed.

In preferred embodiments, the pressure ulcer prevention tool in accordance with an aspect of the invention is a system for detecting patient movement and recording the same comprising: a strap comprising a horizontal component and at least two vertical components, said horizontal component having a closure mechanism to secure around an object, and said at least two vertical components, each attaching to said horizontal component in two locations; one or more sensors positioned on the horizontal component of said strap, said one or more sensors each comprising one or both of an accelerometer and a gyroscope, each said accelerometer determining the acceleration caused by a patient's movement to said sensor, and said gyroscopes determining the position of a patient in relation to said sensor; wherein the horizontal strap transfers forces from a patient to each of the sensors.

In certain embodiments, the object is a bed having a head and foot ends, and said horizontal strap is secured around one end of the bed, with the vertical straps engaging over the head end of the bed, and wherein the sensors are positioned between one and three feet from the head end of the bed.

In certain embodiments, the horizontal strap that connects the two sensors at the head of the bed is configured to transfer force from the patient to the sensors and to deflect the gyroscopes when the patient moves.

In a preferred embodiment, the pressure ulcer prevention tool in accordance with an aspect of the invention is a system for monitoring the movement of a patient to prevent pressure ulcer formation comprising: an accelerometer and a gyroscope, suitable to detect movement of a patient on a bed, said system utilizing data from said accelerometer and gyroscope within an algorithm running on a processor or other logic to identify and calculate the need for a patient to be moved, said system comprising a display and a timer, said display visually displaying said timer; wherein said algorithm modifies the timer on said display when the accelerometer or gyroscope identifies movement of said patient, with no two hours elapsing on said timer, until a health care provider is signaled to ensure the patient has been turned. In a particular embodiment, detection of a patient's motion using the accelerometers is used to identify the movements of the patient, wherein movements will increase the time needed until the patient is turned. In preferred embodiments, upon expiration of the timer, the system is configured to alert said care giver to turn said patient.

In preferred embodiments, the accelerometer and the gyroscope of the described systems measure the movements of a patient over the course of a time period and compare the patient's movements recorded by said accelerometer and gyroscope to a predetermined set of movements and compare if the patient is moving a below average, average, or above average amount as compared to the predetermined set of movements. In determining a predetermined set of movements, use can be made of historical averages of movement for this specific patient to determine if the patient is moving more or moving less than they were before, by comparing the current amount of movement to the historical averages. In some cases, this can be used as a proxy for whether the patient's health is improving.

A further embodiment is directed towards a timing system for patient monitoring comprising: a sensor, a sensor strap, a display, and a computer implemented processor for processing sensor information: wherein, said sensors detect movements of a patient and collect information for processing, wherein the information collected by the sensors is processed by an algorithm, for example, running on the processor, configured to assist healthcare workers in preventing pressure ulcers and the relevant information displayed on the display; said system further generating said processed information to at least one computing device, wherein the availability of the information at the bedside and in a centralized location will assist healthcare providers to act intelligently and quickly in pressure ulcer prevention.

A further embodiment is directed towards a method for preventing pressure ulcers comprising: identifying a patient being positioned in a bed, said positioning being detected by one or more sensors, and an accelerometer, and a gyroscope, oriented on a horizontal strap attached to the head end of a bed; detecting the movements of a patient in a bed, wherein vibrations on said horizontal strap engage the accelerometer and/or gyroscope; wherein upon identification of said patient in said bed, beginning a timer counting down from two hours time; modifying the timer to increase the time when the sensors detect a movement of the patient from one position to a second position; and moving the patient to another position upon the expiration of the timer.

In preferred embodiments, the method further includes modifying the timer to increase the time when the accelerometer measures at least a certain number of movements exceeding a minimum threshold number of movements, such as, for example, 10 movements, within a predetermined amount of time. In preferred embodiments, the predetermined amount of time may be, for example, between 1 minute and 60 minutes, although the invention is not limited to this amount of time.

In a further embodiment, a method for timing the movement of a patient in a hospital bed comprises: detecting the presence of a patient in said bed in a first position, said detection being identified by one or more sensors, said one or more sensors positioned on a horizontal strap attached to said hospital bed; starting a timer having a duration of two hours from the moment of detection of the presence of the patient in said bed; generating an alarm at the expiration of two hours; identifying the movement of said patient through said sensors and upon detection of a second position of said patient, starting a second timer having a predetermined time period duration, e.g. two hours. In a preferred embodiment, after the second timer has begun, detecting the movement of said patient to said first position, reducing the time to the time that has elapsed since the second timer has begun.

### BRIEF DESCRIPTION OF THE DRAWINGS

FIG. 1 depicts a bed comprising the pressure ulcer prevention tool (“the device”) set up for use. Depicted is a hospital bed frame 1 where the device can be advantageously installed. In the illustrated embodiment, the device includes the following components: the vertical component of the sensor strap 2, the horizontal component of the sensor strap 6, the IMU sensors 3 and 4 embedded into the strap, and the bed-mounted display 5.

FIG. 2 depicts the foot of the bed where the bed-mounted display is attached. The bed-mounted display can be attached elsewhere in the patient room if the foot of the bed is not a suitable mounting point.

FIG. 3 depicts a closer view of the head of the bed where the strap and sensors are positioned. The vertical strap 2 component of the sensor strap helps to align the strap in the correct vertical position, wherein the horizontal strap component 6 of the sensor strap helps to hold the IMU sensors 3 and 4 against the bed and transfer motion from the patient to the IMU sensors.

FIG. 4 depicts the bed-mounted display 5, which, in the illustrated embodiment, includes a rotating bezel for user interface navigation, a textured grip for easy rotating, lights in the bezel for user notification, and a screen for displaying color, graphical indicators, and text.

FIG. 5 depicts the user interface of the bed-mounted display. The first primary screen shows a counting down of the amount of time until the next time a patient must be turned. As the time counts down, the circular graph will deplete and the color of the circular graph will change to indicate how close the turn timer is to reaching a time of zero. The graph is important because it provides the user a sense of scale for how much time is left in the countdown. The colors that the graph will use are, for example, green, yellow, and red. The second primary screen (below the first primary screen) will be presented if the patient turn is overdue and counts up to indicate by how long the turn is overdue. The secondary screens are accessed by rotating the bed-mounted display bezel and show additional information. In the illustrated embodiment, the secondary screens show: “Average Time Between Turns”, “Time Spent Inclined Today”, and a percentage of time the patient has spent in the “Center”, “Right”, and “Left” position over the last 24 hours.

FIG. 6 is a block diagram that schematically shows the electronic components of the system in communication with the central repository.

### DETAILED DESCRIPTION OF THE EMBODIMENTS

The device described herein improves patient care and reduces and prevents the formation of pressure ulcers. The device advantageously comprises (A) one or more sensors (for example Inertial Measurement Units (IMUs) which consist of accelerometers, gyroscopes, and magnetometers) mounted to the head of the bed using a specialized strap that transfers motion from the patient to the sensors, (B) an algorithm, (C) an intuitive bed-mounted display that delivers information to the healthcare provider in three forms, each tailored to deliver information at varying distances and to reinforce the information delivered by the other forms, and (D) a central repository of information that allows a health care professional to view the pressure ulcer related patient information from a remote location.

This device will aid nurses and patient care givers in ensuring that the patient is receiving proper care and is being turned at an appropriate rate to reduce the chance of formation of pressure ulcers.

A first issue that this device solves is for automatic and accurate logging of patient turns. Although some nurses document when they have performed a patient turn immediately after the turn has occurred, the majority of nurses will perform all of their documentation, including turning documentation, during breaks. In the latter case, nurses will likely report that they turned the patient on time at whatever the pre-determined interval was. This leads to inaccuracies in when patient turns actually occurred and the actual duration between turns. The disclosed device solves this problem by detecting exactly when a turn has occurred and automatically logging it. This will allow a nurse and hospital administration to have a daily and historical log of exactly when turns occurred and the duration between turns.

The device will further aid nurses by eliminating the need for nurses having to rely on their memory for when a patient needs to be turned. In most hospital settings nurses will decide when to turn a patient based on when they remember turning the patient last. While this might be feasible when a nurse is working with a few patients, it is not scalable. The device solves this problem by automatically detecting when a turn occurred and displaying a timer for when the next turn needs to occur on both a bed-mounted display and a remote dashboard visible by nurses. The bed-mounted display utilizes three forms of information delivery to inform the nurse of the patient turn status from variable distances. The three forms are (1) color, (2) graphics, and (3) text. The screen and casing of the bed-mounted display will light up varying colors to indicate how far along the patient turn timer is. Color serves the purpose of delivering information when the nurse only has the bed-mounted display in their peripheral vision and acts to quickly reinforce information when the nurse is looking directly at the bed-mounted display. As the nurse approaches the device and is directly looking at it, the graphics become more apparent and will provide more granular information than the color indicators. Finally, when the nurse is close to the device, the text on the bed-mounted display will provide the nurse the most granular information. Even at this close proximity, the color and graphics help reinforce the information and allow the nurse to make quick decisions. Exactly what information the bed-mounted display will display is described elsewhere herein, including in the description of drawings.

Furthermore, the device will provide nurses with new information that was previously not available to them. Indeed, unless a nurse is in the patient's room, or a care giver is actually with the patient, they will not have a complete understanding of the movements of the patient from a Time 1 to a Time 2. Furthermore, even when a nurse or a care giver is in the patient's room, they still may not be able to determine whether the patient has shifted weight or positions in a subtle manner that relieves pressure stress. Ultimately, nurses and care givers do not know about the patient's position and movements history in a detailed manner that can be used to prevent pressure ulcer risk.

Risk of developing a pressure ulcer is effected by the patient's position in bed and by patient mobility. One important position that increases risk of pressure ulcer development is if the patient is inclined in bed. Because the patient and patient's family can often control the position of the bed, the nurse does not always know when a patient has been inclined and, if so, for how long. Using gyroscopes the device can detect at what angle the patient is inclined and for how long. This information can be displayed on the bed-mounted display and on the remote dashboard. The nurse can use this information to educate the patient about the risks of being in an inclined position or to adjust the frequency of patient turns. The device, using the sensors discussed above, can also take inclination data and directly change the turn timer. If a patient has been inclined for a long period of time the device can turn the timer forward causing turns to occur more frequently. Immobile patients have an increased risk of pressure ulcer development. There is currently no adequate way to quantify all of a patient's movements in a bed over the duration of the patient's hospital stay. Using accelerometers the device can detect the patient's movement in bed and can report the patient's motion as a comparison to an average patient's motion in a bed, and/or can directly change the turn timer based on the patient's motion. If a patient is very mobile, their turn timer can be turned back and turns can occur less frequently. If a patient is immobile their turn timer can be turned forward and turns can occur more frequently. Indeed, the precise movements allow for modification of the required time for a patient turn. Accordingly, more active patients can extend the time necessary for a turn, while those who remain essentially motionless, will require turns at the normal prescribed time, typically two hours.

If a patient has prematurely aborted a turn, the nurse does not know when that occurred. When a nurse turns a patient to one side, the patient has to stay in that turned position for the duration of that turn cycle to receive the complete pressure relief. However, some patients may be non-compliant due to varying factors. This can cause them to prematurely abort a turn after the nurse has left the room. Because the nurse only visits a hospital room at set intervals he/she will not be able to tell how long a patient has had pressure relief if the patient aborts the turn early. Using gyroscopes within the sensors 3 and 4, the device can detect if a turn has occurred and how long the patient is in the turned position. The device will be able to alert the nurse if the patient has prematurely aborted the turn and can turn the timer back to its pre-turn amount if the patient aborts a turn within a set amount of time after the nurse performed the turn.

Indeed, this feature is important to ensure that a small movement, or even a large movement does not reset the timer, when the patient quickly moves back to a prior position. Furthermore, even where a patient is turned, should the patient return to the prior position, the device can detect a movement and provide an appropriate reduction in time before another turn, or alert a care giver of an aborted turn.

Finally, because each patient is different, and turns are performed manually, until now no system has provided for a central location that identifies data relevant to each patient with regard to their status with regard to pressure ulcer formation.

In a normal hospital setting there previously has been no way for a nurse to see a live view of every patient's turn status. Using the dashboard feature of the device, a nurse will now be able to see the current turn timer and other relevant information for each patient whose bed, or wheelchair, is equipped with the device. This will help to optimize a nurse's schedule by helping them to determine exactly what patient's room needs attention, and when such attention is needed. The dashboard can be configured to display, for example at a central location, information from all of the occupied equipped beds in a hospital, a specific hospital floor, and an individual patient.

There is also a concern for nurses and care givers as family members may have the best intentions of helping the patient. However, family members do not have concrete evidence about their patient's turns and thus may provide inaccurate or conflicting accounts of what is necessary for appropriate patient care.

In the case where a family member is concerned that their patient is not being turned at an adequate frequency the device can act as a concrete piece of evidence that they can discuss with the nurse or physician when they are advocating for their patient to be turned. In addition, the bed-mounted display will show family members that turns are occurring at the correct times, which can provide them with emotional support.

The device is also helpful for care by non-professional patient care staff. For example, family members who are turning their patient do not have feedback to indicate if turn was done correctly. Family members or other untrained professionals who are tasked with turning a patient may not know when they have correctly turned a patient. Because the device has the ability to detect when a turn has occurred, it can give the person turning the patient an audio and visual indication that the turn has been performed correctly and the patient is stable in the correct turn position.

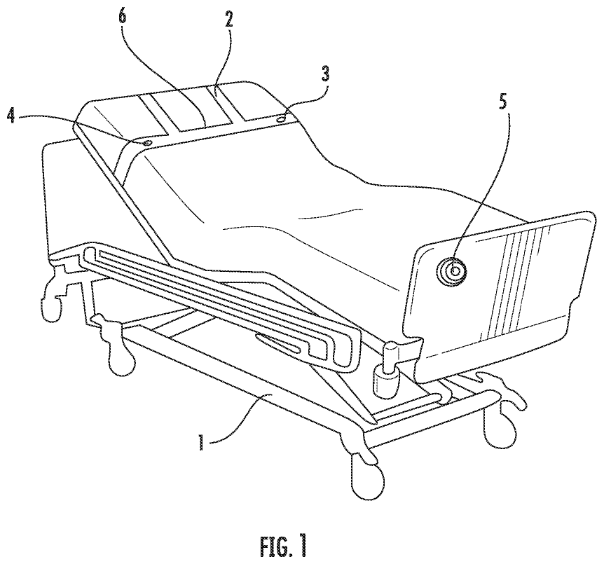

In a preferred embodiment, FIG. 1 depicts a hospital bed 1, having the device provided, with a sensor unit, comprising, for example, sensors 3 and 4, and a local display unit 5. The strap is made up of one horizontal strip 6 of material and two vertical strips of material 2 that are attached to the horizontal strip. The horizontal strip of the strap is adjustable so that the strap can accommodate different sized mattresses. It is secured in the horizontal position using a “buckle with slider bar”. Alternatively the horizontal strip of fabric can contain elastic material on the underside that allows the strap to be stretched in the horizontal axis and will squeeze the bed once positioned, or other securing mechanism, as known to a person of ordinary skill in the art. This design helps the healthcare professional position the strap and sensors in the correct location on the bed by limiting the vertical position that the strap can be positioned in. This design is also key to the strap being easy to clean, because it is made of strips of durable material, preferably nylon. However additional suitable materials that may be used include cotton and other synthetic or natural woven or non-woven materials. The straps, instead of being a solid cover that goes over the head of the bed, allows a nurse or member of the sanitation crew to easily clean the top and underside of the strap. The IMU sensors 3 and 4 are embedded into the strap. This is important because the horizontal component of the strap 6 helps to transfer patient motion to the IMU sensors. Indeed, the movement along the length of strap 6, or even the vertical straps 2, imparts motion, vibrations, or other movements to the sensors 3 and 4. However, while there are advantages to having the sensors 3 and 4 be embedded in the strap, the invention is not limited to this embodiment. Thus, the sensors 3 and 4 could, for example, be affixed to the bed by an adhesive, or in other ways known to those skilled in the art. The bed-mounted display 5 is positioned in a location where the information can be easily seen by healthcare providers, and can selectively be placed where appropriate. If the sensors 3 and 4 are affixed by adhesive or in another manner, it is not necessary to have the vertical straps, since the horizontal straps will be all that is needed, the horizontal component of the straps will be directly adhered to the bed.

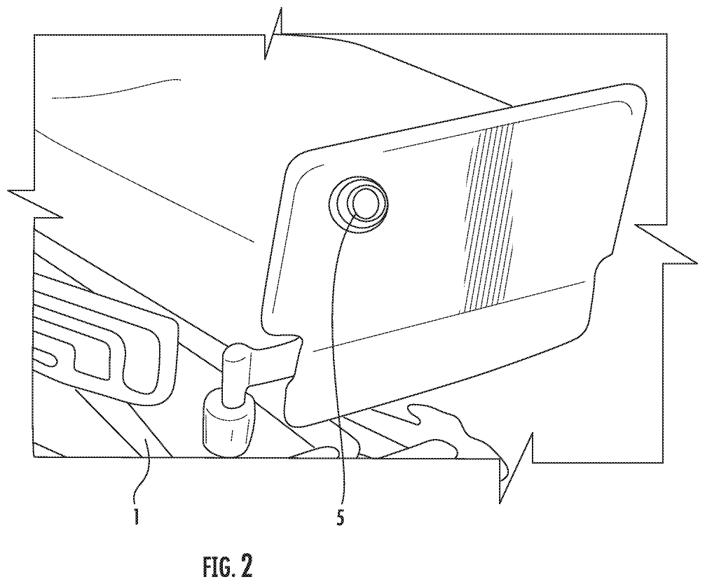

FIG. 2 depicts the foot of the bed 1 where the bed-mounted display 5 is attached. The bed-mounted display 5 preferably has a modular mounting system in that it can instead be attached elsewhere in the patient room if the foot of the bed is not a suitable mounting point. This modular mounting system allows the display 5 to work with different types of hospital beds and surfaces that the display was not initially configured to mount on. The only requirement for the bed-mounted display location is that it is in a location that the healthcare provider can see the information that it is displaying.

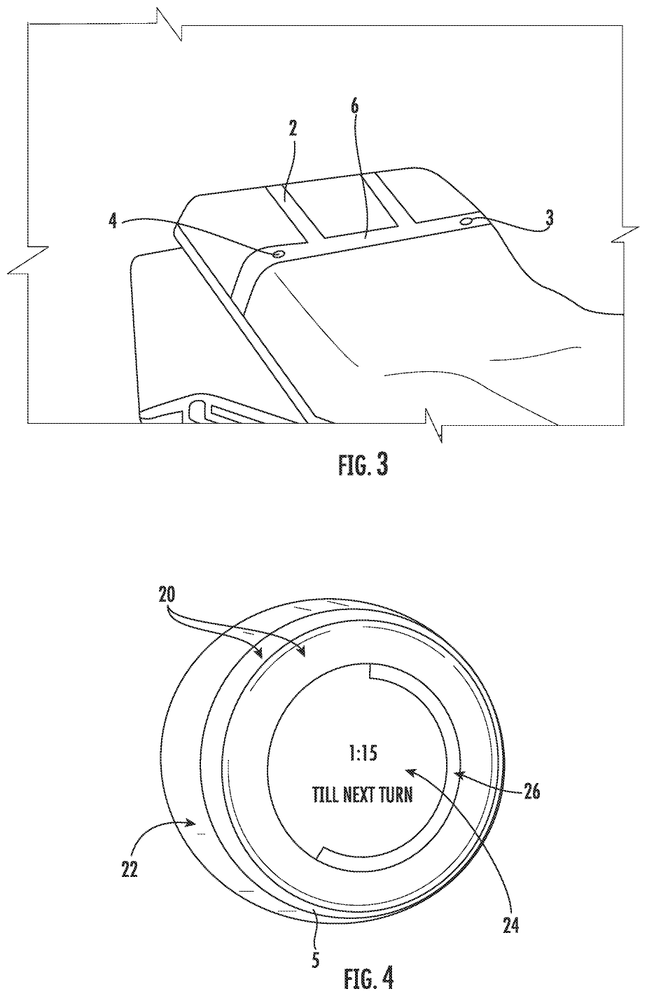

FIG. 3 depicts a closer view of the head of the bed where the device strap and sensor are positioned. The overall design of the straps helps a user to position the device correctly. The vertical strap 2 component of the sensor strap 6 helps to align the strap in the correct vertical position by limiting the extent that the strap can be pulled down in the vertical direction. The horizontal strap component of the sensor strap 6 helps to hold the IMU sensors 3 and 4 against the bed and transfer motion from the patient to the IMU sensors. The sensors 3 and 4 are attached or embedded in the strap near the edge of the bed where they will not be uncomfortable for the patient. The sensors 3 and 4 are able to be placed further away from the patient because the horizontal strap 6 helps to transfer the patient's motion to the sensors 3 and 4. Preferably the straps are made of relatively thin material to allow the sensor mount on the straps to be cleaned easily.

FIG. 4 depicts an exemplary embodiment of the bed-mounted display 5. The display 5 contains a rotating bezel 20 for user interface (UI) navigation, a textured grip 22 for easy rotating user interaction and to provide a visual indication that the device is meant to be touched and the bezel 20 is meant to be rotated. Lights in the bezel 20 for user notification serve the purpose of providing user notification along with all of the information provided by the screen 24. The screen displays color, graphical indicators, and text. The graphical indicator 26 provides the user with a sense of scale and can provide information when the user is too far away from the display 5 to read the text information shown on the text indicator 24, which provides the most granular form of data. The text information shows, for example in numerical digital form, how long until the next scheduled turn of the patient.

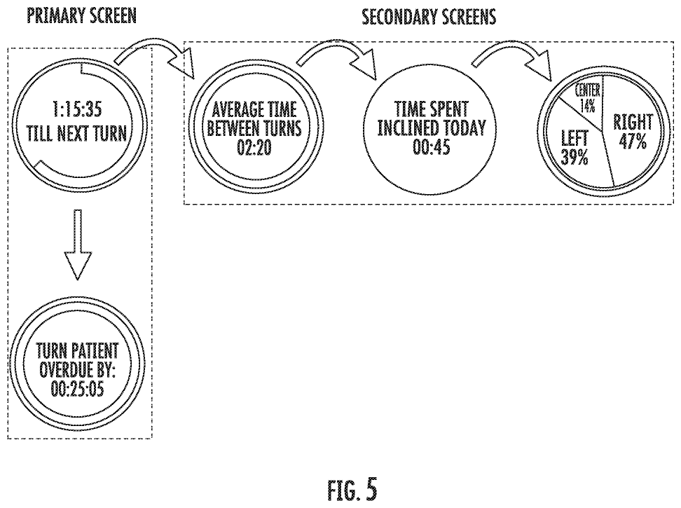

FIG. 5 depicts the user interface of the bed-mounted display. The first primary screen counts down to indicate to how much time until the next turn. As the time counts down the circular graph at the periphery will deplete and the color of the circular graph will change to indicate how close the turn timer is to reaching a time of zero. The graphical representation is important because it gives the user a sense of scale for how much time is left in the countdown. The colors that the graph uses are preferably green, yellow, and red, although other colors, or even patterns, may be used. The second primary screen, i.e., the screen below the first primary screen in FIG. 5, will be presented if the patient turn is overdue and counts up to indicate by how long the turn is overdue. The secondary screens are accessed by rotating the bed-mounted display bezel and show additional information. Examples of such additional information, as shown in the figure, are “Average Time Between Turns”, “Time Spent Inclined Today”, and a pie chart type graph showing percentage of time the patient has spent in the “Center”, “Right”, and “Left” position over the last 24 hours. This is only one example of a graphic user interface. Others are readily known.

Additional features of the bed-mounted display include the ability to be activated in any known manner, such as a touch screen or button, and also provides navigation controls such as touch panel controls. If the, e.g., button, etc., is pressed a single time, the device will return to the primary screen if it was on a secondary screen. If the button in pressed and held for a few seconds the device will enter an edit menu by which the user can change settings such as the time interval between turns on, the patient assigned to the unit, and have the option to reset to system. If the button is double pressed quickly a screen will come up that will let the nurse log patient non-compliance if the patient has refused a turn. The nurse will have to double press the button to enter the non-compliance screen and then rotate the bezel 180°. Making the nurse turn the bezel 180° can be used as a security feature of the device to ensure that non-compliance is not accidentally logged. Accordingly, the display 5 can provide access to menus, displays, and the like by activating the control mechanism through compression of the display.

Alternatively, an electric button or toggle may be activated in another position to provide for an access or menu like button. Those of skill in the art will recognize that provided such an electronic switch is routine in the art.

In preferred embodiments the sensors 3, 4 are multi-function sensors, comprising a gyroscope and accelerometer within the same IMU unit. These multi-function sensors may be placed in one of several places. For example, by placing the sensors 3 and 4 at the shoulder level and on the top surface of the bed provides the best data. Placing the sensors on the side of the mattress provides sufficient data, but not as clear. Placing the sensor above the patient's head provides some data, but the signal is inadequate. Part of what makes the device operate effectively at the level of the shoulder is that in that location the patient is lying on the strap 6, with allows transfer of motion directly to the sensors 3 and 4. Thus, optimal sensor placement of the sensors at the shoulder level and on the top surface of the bed is optimal.

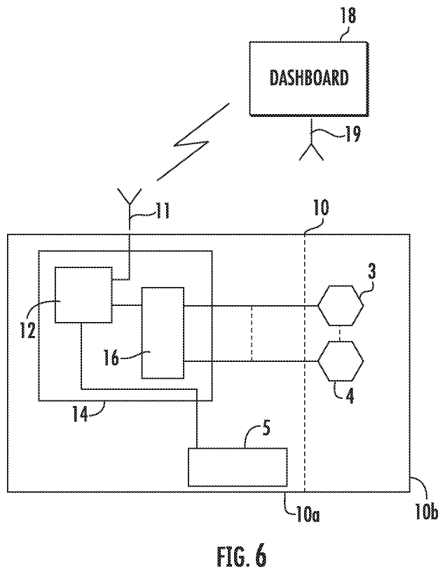

FIG. 6 schematically shows the electrical connections relating to an exemplary embodiment of the pressure ulcer prevention tool 10 (the device 10) showing one or more sensors 3, 4, which may each include one or more of accelerometers, gyroscopes, and magnetometers, that conveys the sensor data to a controller 14 by depositing the data in memory 16 of the controller 14. As discussed above, a specialized algorithm is executed, for example as a computer program running on a processor 12 of the controller 14 of the device 10. In a preferred embodiment, the device 10 has two portions. A portion 10a is located in the housing that contains the display 5 and includes the display 5 itself, the controller 14, the memory/database 11, the processor 12, and the transceiver 11. A portion 10b consists of the sensors 3, 4 which communicate with the portion 10a by electrical connection, either wired, or wireless.

The controller 14 thus configured takes the data collected by the IMUs, stores it in the memory 16 of the controller 14 and produces actionable information for the prevention of pressure ulcers. The intuitive bed-mounted display 5, discussed in detail above, delivers information to the healthcare provider in multiple forms, for example three forms, each tailored to deliver information at varying distances and to reinforce the information delivered by the other forms. The device 10 of the bed 1 is configured to communicate with a central repository 18 of information, referred to below as a “dashboard”, that allows a health care professional to view the pressure ulcer related patient information from a remote location. The memory 16 also stores a program or other logic that effects the algorithm by running the program on the processor 12. The device 10 also includes a transceiver 11 to communicate, e.g., wirelessly, with the dashboard 18, which itself has a transceiver 19 for receiving data wirelessly.

While FIG. 6 shows the device 10 communicating remotely to a dashboard 18, the device 10 can also function independently, using only its onboard display 5 for indication, without transmitting data to a central remote location dashboard 18. Also, while FIG. 6 shows a wireless connection between the device 10 and the dashboard 18, alternatively, the device(s) 10 can be configured to connect to the dashboard 18 in a wired manner, e.g., over the hospital's wired Internet connection.

According to one aspect of the invention, the system can be reset if the patient has not been in the bed for a predetermined period of time to ensure that the device is not used for a new patient without being set up for that new patient.

Sensor Mounting

It is important for the horizontal strap 6 to be tight around the mattress and for the sensors 3 and 4 to be located at the correct bed position. The horizontal strap 6 being tight around the mattress allows for the strap to transfer motion to the sensors 3 and 4 that are embedded in the strap. To ensure that the strap is tight around mattresses of varying sizes a buckle or elastic can be used. Both of these methods function well. Since the location of the sensors is important, the strap is preferably configured to look like “overalls” so that all the nurse has to do is pull the straps down on the top of the bed all the way, i.e., as far as they will go, and the sensors will be in the correct position.

Patient Position in Bed

Different Sized and Weighted Patients and Patient Placement

The system is configured to input height and weight of the particular patient into the system. Furthermore, the sensors can gather data with regard to particular forces with regard to the placement. Furthermore, the sensors permit detection of the shape of the signal provided by the sensor versus the absolute values of the signal. This can help to evaluate the shape, height, weight, and position of a patient.

Strap Cleaning

The “overalls” design and use of impermeable materials allows a nurse or member of the sanitation crew to easily clean the top and underside of the strap, and replace it in the correct position for further use.

Number of Sensors

Using two sensors provides good information about turn directionality, reduces noise, and allows the system to work better with mattresses that do not deform significantly, such as foam mattresses. Foam mattresses provide a particular challenge because they absorb more of the force of a patient's movement then a traditional spring mattress would. Because of this, if a patient is turning in one direction the sensor that they are turning away from may not experience enough deflection to adequately determine the patient's motion. However, the sensor located in the direction the patient is moving toward will still experience the force of the patient's motion. However, the invention is not limited to the use of two sensors. The device may be able to use only one sensor, or more than two sensors, with appropriate treatment by the algorithm of the input signals.

Bed Display Mounting

Mounting of the display may be in nearly any location at or adjacent to the bed. The goal of the mounting is to allow for appropriate visual for the nurse or caregiver. In certain embodiments, there is a mounting dock that can be secured to nearly any surface to allow for limitless positioning of the display, based on the needs of the care giver.

Quantifying Micro-Motion

The device is configured to detect the patient's movement in bed using accelerometers and gyroscopes. Accelerometers are the primary sensor used for motion detection because they can detect small instantaneous acceleration events. Detection of a patient's motion would rely on determining how much typical patients move. By comparing the patient whose bed is equipped with the device to the movement profile of the average patient in their demographic the device can alert nurses if the patient is moving a below average, average, or above average amount compared to other patients in their demographic. In a more advanced iteration of the device, the patient's movement profile can be used to automatically change the duration of the patient's turn timer. If a patient is very mobile their turn timer can be turned back and turns can occur less frequently. If a patient is immobile their turn timer can be turned forward and turns can occur more frequently. In addition to general patient movement, because there are typically two IMU sensors positioned on the patient's bed the device can determine which direction the patient is moving as well. This could provide healthcare workers with information that would inform them that the patient is only moving in one direction and not providing pressure relief on one side of their body. In addition to providing more information for patients already determined to be at risk for pressure ulcers, if the device is equipped to the bed of a patient previously determined not to be at risk for pressure ulcers, it can detect if the patient's motion has fallen below a predetermined amount which might put them at risk of developing pressure ulcers. Using sensors to monitor a patient's motion in bed is a novel way to determine their risk of developing a pressure ulcer.

Additional features may be included into the device. For example, light sensors or sound sensors can be easily added to the system. The light sensor may comprise an ambient light sensor, and may be used, for example to adjust the brightness of the display or change from blue light to non-blue light wavelength based on the time of day. The ambient light sensor, which may be mounted into the bed-mounted display, will permit detection of how bright the room is and adjust the brightness of the screen and indicator LEDs so that we do not disturb the patient when all the lights in their room are off/dim.

Sound sensors may be used to, for example, wake the display, as it may time out, to allow for dark conditions. Accordingly, a noise or spoken instruction may illuminate the display or increase its intensity. Sound sensors, e.g., microphones, can also be used to adjust the volume of sound that the display makes. For example if the room is very quiet then the volume the device makes can be decreased.

The device provides for a comprehensive monitoring system for evaluating patient position in bed and can be used for many other medical applications. Thus, while the device is discussed above in the context of helping healthcare staff prevent pressure ulcers, the device can be used in other hospital like settings for preventing of other diseases or disorders. One such application is the preventions of Deep Vein Thrombosis (DVTs). DVTs are another medical condition that can be caused by a patient's lack of mobility in a bed. The patient position detection performed by the device can also be used to alert healthcare staff if a patient at risk for developing a DVT by informing the staff members if the patient's mobility is below the average mobility profile for a person in their demographic.

Further applications may include patients at risk for seizure, or for other controlled or uncontrolled bodily movements. The device provides for sensors 3 and 4 that easily identify these movements and allows staff or medical care to properly react to or monitor the movement of a patient from afar. Other medical conditions that may be detected are those related to inclination of the patient in the bed. For example, people with heart conditions, like congestive heart failure, pericardial effusion, and constrictive pericarditis, can have a condition called orthopnea. Those suffering from this condition have trouble breathing when lying flat and they have to sleep, and, in general remain, in an inclined position in order to breathe comfortably. The inclination detection function of the device can be used to keep a historical record of the inclination the patient needs to be at to breathe comfortably. This inclination can be used as a proxy for how their heart condition is progressing, i.e., the more inclined in bed being worse and less inclined in bed being better.

Those of skill in the art will recognize that the device maybe modified by those of skill in the art. Certain attachment mechanisms for attaching sensors, for attaching the straps and the like are omitted, as these are routine and understood by an ordinary skill person. However, additional modifications may be provided without modification from the spirit of the invention.

What is claimed is:

1. A system for detecting patient movement and recording the same, comprising:
a strap comprising at least a horizontal component, said horizontal component having a closure mechanism to secure around an object, and;
one or more sensors positioned on the horizontal component of said strap, said one or more sensors each comprising at least one of an accelerometer and a gyroscope, each said accelerometer determining acceleration caused by a patient's movement, and each said gyroscope determining the position of a patient in relation to said one or more sensors,
wherein the horizontal strap transfers forces from a patient to each of the one or more sensors.

2. The system of claim 1, further comprising at least two vertical components, said at least two vertical components attaching to said horizontal component in two respective locations

3. The system of claim 2, wherein the object is a bed having a head and foot ends, and said horizontal strap is secured around one end of the bed, with the vertical straps engaging the head end of the bed, and wherein the sensors are positioned between one and three feet from the head end of the bed.

4. The system of claim 1, wherein the horizontal strap that connects the one or more sensors at the head of the bed transfers force from the patient to the one or more sensors and to deflect the gyroscopes when the patient moves.

5. A system for monitoring the movement of a patient to prevent pressure ulcer formation, comprising: an accelerometer and gyroscope, configured to detect movement of a patient on a bed, said system comprising a controller configured to utilize data from said accelerometer and gyroscope by execution of an algorithm in logic of the controller, the controller being configured to identify and calculate the need for a patient to be moved, said system comprising a display and a timer, said display visually displaying said timer; wherein said controller is configured to modify the timer on said display when the accelerometer or gyroscope identify movement of said patient, with no predetermined time interval elapsing on said timer, until a health care provider is signaled to ensure the patient has been turned.

6. The system of claim 5, wherein detection of a patient's motion using the accelerometers identifies the movements of the patient, and detection of such movements increases the time needed until the patient is turned.

7. The system of claim 5, wherein upon expiration of the timer, the system controller alerts said care giver to turn said patient.

8. The system of claim 5, wherein the accelerometer and the gyroscope measure the movements of a patient over the course of a time period and the controller compares the patient's movements recorded by said accelerometer and gyroscope to a predetermined set of movements to determine if the patient is moving a below average, average, or above average amount as compared to the predetermined set of movements.

9. A timing system for patient monitoring, comprising: a sensor, a sensor strap, a display, and a computer implemented processor for processing sensor information: wherein, said sensors detect movements of a patient and collect information for processing, wherein the information collected by the sensors is processed by the processor programmed to effect the algorithm so as to assist in preventing pressure ulcers and display the relevant information on the display; said system further transmitting said processed information to at least one computing device located in a centralized location remote from the timing system, wherein the viability of the information at a bedside and in the centralized location is configured to assist healthcare providers to act to prevent pressure ulcers.

10. A method for preventing pressure ulcers, comprising:
identifying a patient being positioned in a bed, said positioning being detected by one or more sensors, each having at least one of an accelerometer and a gyroscope, said one or more sensors being arranged on a horizontal strap attached to a head end of the bed;
detecting movements of the patient in the bed, wherein movements on said horizontal strap engage the accelerometer and/or gyroscope;
wherein upon identification of said patient in said bed, beginning a timer counting down from two hours time; modifying the timer to increase the time when the one or more sensors detect a movement of the patient from a first position to a second position;
moving the patient to another position upon the expiration of the timer.

11. The method of claim 10, wherein modifying the timer to increase the time is performed when the accelerometer measures at least ten movements within a predetermined amount of time.

12. The method of claim 11, wherein the predetermined amount of time is between 1 minute and 60 minutes.

13. A method for timing the movement of a patient in a hospital bed, comprising:
detecting the presence of a patient in said bed in a first position, said detection being identified by one or more sensors, said one or more sensors positioned on a horizontal strap attached to said hospital bed;
starting a timer having a duration of two hours from the moment of detection of the presence of the patient in said bed;
generating an alarm at the expiration of two hours;
identifying the movement of said patient through said sensors and upon detection of a second position of said patient, starting a second timer having a duration of two hours.

14. The method of claim 13, wherein after the second timer has begun; detecting the movement of said patient to said first position, and reducing the time to the time that has elapsed since the second timer has begun.
